(19) United States
(12) Patent Application Publication
(10) Pub. No.: US 2025/0191786 A1
(43) Pub. Date: Jun. 12, 2025

(54) AI-BASED DRUG SIDE EFFECT PREDICTION

(71) Applicant: DEEP FOREST SCIENCES, INC., Palo Alto, CA (US)

(72) Inventors: BHARATH RAMSUNDAR, Berkeley, CA (US); SANDYA SUBRAMANIAN, Berkeley, CA (US)

(73) Assignee: DEEP FOREST SCIENCES, INC., Palo Alto, CA (US)

(21) Appl. No.: 18/972,698

(22) Filed: Dec. 6, 2024

Related U.S. Application Data
(60) Provisional application No. 63/606,864, filed on Dec. 6, 2023.

(51) Int. Cl. G16H 70/40

(52) U.S. Cl. CPC G16H 70/40

(57) ABSTRACT

Apparatuses, methods, program products, and systems are disclosed for AI-based drug side effect prediction. An apparatus is configured to determine molecular structure information for one or more molecules, predict one or more potential side effects based on the molecular structure information using a machine learning model, and provide the predicted one or more potential side effects to a user.

### CROSS REFERENCE TO RELATED APPLICATIONS

This application claims the benefit of U.S. Provisional Patent Application No. 63/606,864 entitled BUILDING AI MODELS OF PATIENT-SPECIFIC DRUG SIDE EFFECT PREDICTIONS and filed on Dec. 6, 2023, for Bharath Ramsundar et al., which is incorporated herein by reference.

### FIELD

This invention relates to artificial intelligence (AI) and more particularly relates to AI-based drug side effect prediction.

### BACKGROUND

Planning for unforeseen side effects of medication when choosing management options for diseases such as cancer is a perennial problem for clinicians. This problem is enhanced for designer diseases in which each patient can present the disease completely differently. Currently, the only information about side effects is from the original clinical trials on the medication in animal and human models.

### SUMMARY

Apparatuses, methods, program products, and systems are disclosed for AI-based drug side effect prediction. In one embodiment, an apparatus is configured to determine molecular structure information for one or more molecules, predict one or more potential side effects based on the molecular structure information using a machine learning model, and provide the predicted one or more potential side effects to a user.

In one embodiment, a system includes a first machine learning model configured to analyze molecular structure information for one or more molecules to create a molecular embedding vector, a second machine learning model configured to analyze electronic health information for one or more patients to create a patient embedding vector, and a third machine learning model configured to analyze a combination of the molecular embedding vector and the patient embedding vector to predict one or more potential side effects.

A method, in one embodiment, includes determining molecular structure information for one or more molecules, predicting one or more potential side effects based on the molecular structure information using a machine learning model, and providing the predicted one or more potential side effects to a user.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating one embodiment of a system in accordance with the subject matter disclosed herein.

FIG. 2 illustrates one embodiment of an apparatus for AI-based side effect prediction in accordance with the subject matter disclosed herein.

FIG. 3 illustrates one embodiment of an apparatus for AI-based side effect prediction in accordance with the subject matter disclosed herein.

FIG. 4 illustrates one embodiment of a system architecture in accordance with the subject matter disclosed herein.

FIG. 5 illustrates one embodiment of a method for AI-based side effect prediction in accordance with the subject matter disclosed herein.

FIG. 6 illustrates one embodiment of a method for AI-based side effect prediction in accordance with the subject matter disclosed herein.

### DETAILED DESCRIPTION

Planning for unforeseen side effects of medication when choosing management options for diseases such as cancer is a perennial problem for clinicians. This problem is enhanced for designer diseases in which each patient can present the disease completely differently. Currently, the only information about side effects is from the original clinical trials on the medication in animal and human models. However, automated models, specifically utilizing the benefits of machine learning, can help advance precision medicine by predicting side effects in silico. Recent advances in applying machine learning techniques to chemical structures of molecules enables this. The subject matter herein provides an early proof-of-concept implementation that transforms molecular structures to a list of potential predicted side effects. An extended architecture that pairs molecular structures with patient profiles is designed to generate patient-specific recommendations.

The use of AI tools to predict side effects for drugs can be a powerful tool for doctors, researchers, developers, or the like. By combining molecular information about drugs with patient specific knowledge, AI methods can predict patient-specific side effects with high accuracy.

Unexpected side effects of medication for diseases like cancer are a major problem for clinicians. For example, chemotherapies can induce a broad range of side effects ranging from fatigue to nausea to altered taste. Adverse drug reactions cost the health care system in the United States more than 30 billion dollars per year. The problem is worsened in designer diseases in which patient presentation and side effects are both highly individual-specific. In Parkinson's disease, for example, standard treatments like levodopa/carbidopa can induce a broad range of dyskinesias with patient-specific profiles.

Designing automated methods to predict drug side effects can be a powerful boon to clinicians that enables precise treatment strategies with fewer adverse drug reactions. The subject matter herein is directed to analyzing a dataset of reported patient side effects of drugs, e.g., drugs that are approved for use by a regulatory body such as the Federal Drug Administration (FDA), building proof-of-concept machine learning models that predict side effects from molecular structures, and proposing a design for a patient-specific prediction engine drawing on electronic health record (EHR) data.

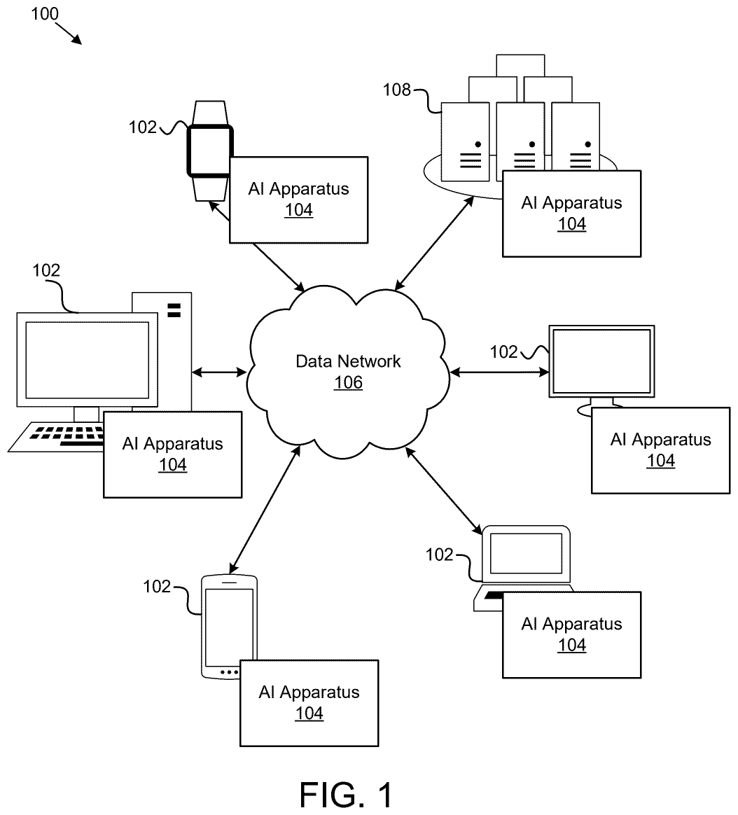

FIG. 1 is a schematic block diagram illustrating one embodiment of a system 100 for techniques for building AI models of patient-specific drug side effect predictions. In one embodiment, the system 100 includes one or more information handling devices 102, one or more AI apparatuses 104, one or more data networks 106, and one or more servers 108. In certain embodiments, even though a specific number of information handling devices 102, AI apparatuses 104, data networks 106, and servers 108 are depicted in FIG. 1, one of skill in the art will recognize, in light of this disclosure, that any number of information handling devices 102, AI apparatuses 104, data networks 106, and servers 108 may be included in the system 100.

In one embodiment, the system 100 includes one or more information handling devices 102. The information handling devices 102 may be embodied as one or more of a desktop computer, a laptop computer, a tablet computer, a smart phone, a smart speaker (e.g., Amazon Echo®, Google Home®, Apple HomePod®), an Internet of Things device, a security system, a set-top box, a gaming console, a smart TV, a smart watch, a fitness band or other wearable activity tracking device, an optical head-mounted display (e.g., a virtual reality headset, smart glasses, head phones, or the like), a High-Definition Multimedia Interface (“HDMI”) or other electronic display dongle, a personal digital assistant, a digital camera, a video camera, or another computing device comprising a processor (e.g., a central processing unit (“CPU”), a processor core, a field programmable gate array (“FPGA”) or other programmable logic, an application specific integrated circuit (“ASIC”), a controller, a microcontroller, and/or another semiconductor integrated circuit device), a volatile memory, and/or a non-volatile storage medium, a display, a connection to a display, and/or the like.

In one embodiment, the AI apparatus 104 is configured to determine molecular structure information for one or more molecules, predict one or more potential side effects based on the molecular structure information using a machine learning model, and provide the predicted one or more potential side effects to a user. The AI apparatus 104 may be located on a single device/system, may be located on multiple different devices/systems that are interconnected (e.g., a device/system that is configured to receive data, a device/system that is configured to process the data using machine learning models, and/or a device/system that is configured to provide results to a user, e.g., via a graphical interface, an application programming interface (API), or the like).

In one embodiment, the AI apparatus 104 facilitates a system to predict drug side effects from molecular structure and constructs a design for a patient-specific model that can make personalized side-effect predictions. The implementation demonstrates that a machine-learned system can learn to make meaningful predictions of potential side effects directly from the molecular structure. Larger datasets and new methods can improve model performance and reduce adverse side effects for patients. Mature AI systems for side effect prediction can prove powerful aids for clinicians, help reduce the number of adverse drug reactions and move one step closer to personalized therapeutic approaches for complex diseases.

As used herein, AI is broadly defined as a branch of computer science dealing in automating intelligent behavior. AI systems may be designed to use machines to emulate and simulate human intelligence and corresponding behavior. This may take many forms, including symbolic or symbol manipulation AI. AI may address analyzing abstract symbols and/or human readable symbols. AI may form abstract connections between data or other information or stimuli. AI may form logical conclusions. AI is the intelligence exhibited by machines, programs, or software. AI has been defined as the study and design of intelligent agents, in which an intelligent agent is a system that perceives its environment and takes actions that maximize its chances of success.

AI may have various attributes such as deduction, reasoning, and problem solving. AI may include knowledge representation or learning. AI systems may perform natural language processing, perception, motion detection, and information manipulation. At higher levels of abstraction, it may result in social intelligence, creativity, and general intelligence. Various approaches are employed including cybernetics and brain simulation, symbolic, sub-symbolic, and statistical, as well as integrating the approaches.

Various AI tools may be employed, either alone or in combinations. The tools may include search and optimization, logic, probabilistic methods for uncertain reasoning, classifiers and statistical learning methods, neural networks, deep feedforward neural networks, deep recurrent neural networks, deep learning, control theory and languages.

Machine learning (ML) plays an important role in a wide range of critical applications with large volumes of data, such as data mining, natural language processing, image recognition, voice recognition and many other intelligent systems. There are some basic common threads about the definition of ML. As used herein, ML is defined as the field of study that gives computers the ability to learn without being explicitly programmed. For example, for predicting traffic patterns at a busy intersection, it is possible to run through a machine learning algorithm/model with data about past or historical traffic patterns, e.g., to train the machine learning algorithm/model. The program can correctly predict future traffic patterns if it learned/trained correctly from past patterns.

There are different ways an algorithm can model a problem based on its interaction with the experience, environment, or input data. The machine learning algorithms may be categorized so that it helps to think about the roles of the input data and the model preparation process leading to correct selection of the most appropriate category for a problem to get the best result. Known categories are supervised learning, unsupervised learning, semi-supervised learning, and reinforcement learning.

(a) In supervised learning category, input data is called training data and has a known label or result. A model is prepared through a training process where it is required to make predictions and is corrected when those predictions are wrong. The training process continues until the model achieves a desired level of accuracy on the training data. Example problems are classification and regression.

(b) In unsupervised learning category, input data is not labelled and does not have a known result. A model is prepared by deducing structures present in the input data. Example problems are association rule learning and clustering. An example algorithm is k-means clustering.

(c) Semi-supervised learning falls between unsupervised learning (without any labeled training data) and supervised learning (with completely labeled training data). Researchers found that unlabeled data, when used in conjunction with a small amount of labeled data may produce considerable improvement in learning accuracy.

(d) Reinforcement learning is another category which differs from standard supervised learning in that correct input/output pairs are never presented. Further, there is a focus on on-line performance, which involves finding a balance between exploration for new knowledge and exploitation of current knowledge already discovered.

Certain machine learning techniques are widely used and are as follows: Decision tree learning, Association rule learning, Artificial neural networks, Inductive logic programming, Support vector machines, Clustering, Bayesian networks, Reinforcement learning, Representation learning, and Genetic algorithms. In certain embodiments, multiple machine learning algorithms may be applied using ensemble learning. As used herein, ensemble learning may refer to a machine learning technique that combines multiple algorithms to produce a single predictive model.

The learning processes in machine learning algorithms are generalizations from past experiences. After having experienced a learning data set, the generalization process is the ability of a machine learning algorithm to accurately execute on new examples and tasks. The learner needs to build a general model about a problem space enabling a machine learning algorithm to produce sufficiently accurate predictions in future cases. The training examples may come from some generally unknown probability distribution.

In theoretical computer science, computational learning theory performs computational analysis of machine learning algorithms and their performance. The training data set is limited in size and may not capture all forms of distributions in future data sets. The performance is represented by probabilistic bounds. Errors in generalization are quantified by bias-variance decompositions. The time complexity and feasibility of learning in computational learning theory describes a computation to be feasible if it is done in polynomial time. Positive results are determined and classified when a certain class of functions can be learned in polynomial time whereas negative results are determined and classified when learning cannot be done in polynomial time.

In certain embodiments, the AI apparatus 104 may include a hardware device such as a secure hardware dongle or other hardware appliance device (e.g., a set-top box, a network appliance, or the like) that attaches to a device such as a head mounted display, a laptop computer, a server 108, a tablet computer, a smart phone, a security system, a network router or switch, or the like, either by a wired connection (e.g., a universal serial bus (“USB”) connection) or a wireless connection (e.g., Bluetooth®, Wi-Fi, near-field communication (“NFC”), or the like); that attaches to an electronic display device (e.g., a television or monitor using an HDMI port, a DisplayPort port, a Mini DisplayPort port, VGA port, DVI port, or the like); and/or the like. A hardware appliance of the AI apparatus 104 may include a power interface, a wired and/or wireless network interface, a graphical interface that attaches to a display, and/or a semiconductor integrated circuit device as described below, configured to perform the functions described herein with regard to the AI apparatus 104.

The AI apparatus 104, in such an embodiment, may include a semiconductor integrated circuit device (e.g., one or more chips, die, or other discrete logic hardware), or the like, such as a field-programmable gate array (“FPGA”) or other programmable logic, firmware for an FPGA or other programmable logic, microcode for execution on a microcontroller, an application-specific integrated circuit (“ASIC”), a processor, a processor core, or the like. In one embodiment, the AI apparatus 104 may be mounted on a printed circuit board with one or more electrical lines or connections (e.g., to volatile memory, a non-volatile storage medium, a network interface, a peripheral device, a graphical/display interface, or the like). The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of the AI apparatus 104.

The semiconductor integrated circuit device or other hardware appliance of the AI apparatus 104, in certain embodiments, includes and/or is communicatively coupled to one or more volatile memory media, which may include but is not limited to random access memory (“RAM”), dynamic RAM (“DRAM”), cache, or the like. In one embodiment, the semiconductor integrated circuit device or other hardware appliance of the AI apparatus 104 includes and/or is communicatively coupled to one or more non-volatile memory media, which may include but is not limited to: NAND flash memory, NOR flash memory, nano random access memory (nano RAM or “NRAM”), nanocrystal wire-based memory, silicon-oxide based sub-10 nanometer process memory, graphene memory, Silicon-Oxide-Nitride-Oxide-Silicon (“SONOS”), resistive RAM (“RRAM”), programmable metallization cell (“PMC”), conductive-bridging RAM (“CBRAM”), magneto-resistive RAM (“MRAM”), dynamic RAM (“DRAM”), phase change RAM (“PRAM” or “PCM”), magnetic storage media (e.g., hard disk, tape), optical storage media, or the like.

The data network 106, in one embodiment, includes a digital communication network that transmits digital communications. The data network 106 may include a wireless network, such as a wireless cellular network, a local wireless network, such as a Wi-Fi network, a Bluetooth® network, a near-field communication (“NFC”) network, an ad hoc network, and/or the like. The data network 106 may include a wide area network (“WAN”), a storage area network (“SAN”), a local area network (“LAN”) (e.g., a home network), an optical fiber network, the internet, or other digital communication network. The data network 106 may include two or more networks. The data network 106 may include one or more servers, routers, switches, and/or other networking equipment. The data network 106 may also include one or more computer readable storage media, such as a hard disk drive, an optical drive, non-volatile memory, RAM, or the like.

The wireless connection may be a mobile telephone network. The wireless connection may also employ a Wi-Fi network based on any one of the Institute of Electrical and Electronics Engineers (“IEEE”) 802.11 standards. Alternatively, the wireless connection may be a Bluetooth® connection. In addition, the wireless connection may employ a Radio Frequency Identification (“RFID”) communication including RFID standards established by the International Organization for Standardization (“ISO”), the International Electrotechnical Commission (“IEC”), the American Society for Testing and Materials® (ASTM®), the DASH7™ Alliance, and EPCGlobal™.

Alternatively, the wireless connection may employ a ZigBee® connection based on the IEEE 802 standard. In one embodiment, the wireless connection employs a Z-Wave® connection as designed by Sigma Designs®. Alternatively, the wireless connection may employ an ANT® and/or ANT+® connection as defined by Dynastream® Innovations Inc. of Cochrane, Canada.

The wireless connection may be an infrared connection including connections conforming at least to the Infrared Physical Layer Specification (“IrPHY”) as defined by the Infrared Data Association® (“IrDA”®). Alternatively, the wireless connection may be a cellular telephone network communication. All standards and/or connection types include the latest version and revision of the standard and/or connection type as of the filing date of this application.

The one or more servers 108, in one embodiment, may be embodied as blade servers, mainframe servers, tower servers, rack servers, and/or the like. The one or more servers 108 may be configured as mail servers, web servers, application servers, FTP servers, media servers, data servers, web servers, file servers, virtual servers, and/or the like. The one or more servers 108 may be communicatively coupled (e.g., networked) over a data network 106 to one or more information handling devices 102 and may be configured to execute or run machine learning algorithms, programs, applications, processes, and/or the like.

The subject matter herein describes the methodology used to construct, train, and use an AI or machine learning model for drug side effect predictions.

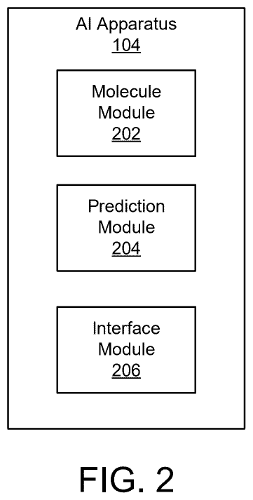

FIG. 2 illustrates one embodiment of an apparatus for AI-based side effect prediction. In one embodiment, the apparatus includes an instance of an AI apparatus 104. The AI apparatus 104, in one embodiment, includes a molecule module 202, a prediction module 204, and an interface module 206, which are described in more detail below.

In one embodiment, the molecule module 202 is configured to determine molecular structure information for one or more molecules. The molecular structure information may include data describing the chemical composition or structure of a molecule such as a molecule of a component of a medicine or drug. In such an embodiment, the molecule module 202 may receive molecular structure information from a data repository, a website, a user, and/or the like.

For example, the molecule module 202 may access a public, remote, or cloud-based data repository, such as the publicly available Side Effect Resource (SIDER) dataset, which may be sourced from the MoleculeNet collection of datasets (MoleculeNet is a benchmark specially designed for testing machine learning methods of molecular properties using different databases of molecules and compounds). The SIDER dataset, for instance, labels molecules with 27 associated MedDRA side effect labels, sourced from the literature using natural language processing (NLP) methods. Table I provides some sample labels.

TABLE I
Sample Side Effects.
Side Effect

Hepatobiliary disorders
Metabolism and nutrition disorders
Musculoskeletal and connective tissue disorders
Gastrointestinal disorders
Immune system disorders

In one embodiment, molecules are represented by text or character strings such as Simplified Molecular Input Line Entry System (SMILES) strings, which is a specification in the form of a line notation for describing the structure of chemical species using short ASCII strings. The molecule module 202, in one embodiment, transforms the SMILES strings into vectorial representations using circular fingerprints. As used herein, circular fingerprints may refer to representations of molecular structures by atom neighborhoods that capture the presence or absence of specific substructures centered around each atom in the molecule. For example, each molecule is represented as a length 1024 sparse bit vector, or alternatively, using a molecular graph representation.

In one embodiment, the molecule module 202 separates each dataset into training/test splits using molecular scaffolds, which group molecules with the same core structure together. Scaffold-based splits may be used to provide a more rigorous test of generalization.

In one embodiment, the prediction module 204 predicts one or more potential side effects based on the molecular structure information using a machine learning model. As used herein, a side effect may refer to a secondary effect of a drug or medical treatment. The machine learning model may include a random forest model, a neural network, a graph-convolutional model, or the like.

In certain embodiments, multiple machine learning models or machine learning ensembles may be used to analyze the molecular structure data. In such an embodiment, the prediction module 204 may use a combination of predictions from a plurality of machine learning models to determine the potential side effects of a drug, based on the molecular structure of the molecules that make up the drug. In one embodiment, the predictions may be associated with a confidence level or value, which the prediction module 204 may use to determine whether to include a prediction of a side effect, e.g., if the confidence value satisfies a predefined threshold.

In one embodiment, the prediction module 204 may provide an interface, such as an API or the like for receiving the molecular structure information (and other data such as electronic health record (EHR) data. In one embodiment, the prediction module 204 may be located on a different system from the molecule module 202 and/or the interface module 206, e.g., as part of a different workflow or pipeline that is connected via a network or other connection.

In one embodiment, the interface module 206 may provide the predicted one or more potential side effects to a user. The interface module 206, for instance, may provide a graphical user interface that presents the predicted one or more potential side effects. In one embodiment, the interface module 206 may provide an API or other programmatic interface for accessing the predicted one or more potential side effects.

In one embodiment, the interface module 206 may generate an explanation of the one or more potential side effects using a large language model. For instance, the interface module 206 may generate and present an explanation of the machine learning models that were used, the data set that was analyzed, the molecular structure information for the drug that was analyzed, and/or the like in an easy to read and understand manner.

In one embodiment, the machine learning model may include a generative AI model or engine. As used herein, generative AI may refer to a type of AI that uses generative models to create new data, such as text, images, videos, and audio. Generative AI models learn the patterns and structures of their training data and use them to produce new data based on input.

In such an embodiment, the interface module 206 presents a graphical element for providing input such as prompts, questions, queries, or the like to the generative AI. The interface module 206 may capture the input and provide it to the generative AI for processing and present the result to the user, which may be formatted as if it were written or developed by a human, e.g., using the large language model or natural language processing.

For example, a user may provide prompts and input such as “Is this person a good candidate for a cancer drug test study,” and provide information about the person. The interface module 206 may receive the input and provide it to the machine learning model, which has been trained on molecular structure information for the drug and patient side effect information and return a result with a prediction of whether the person is a good candidate. Other prompts may include “what are the top five side effects of the drug X,” “what are the potential side effects of a drug with this particular molecular structure?”, and/or the like.

In this manner, the AI apparatus 104 provides a system for accurately analyzing drug information on a molecular level to predict side effects of a particular drug using trained machine learning models. Further, as described below, the AI apparatus 104 may determine side effect predictions for a particular user by analyzing EHR for the user using the machine learning models and the molecular structure data and provide recommendations, suggestions, or the like regarding whether the user should take the drug/medication.

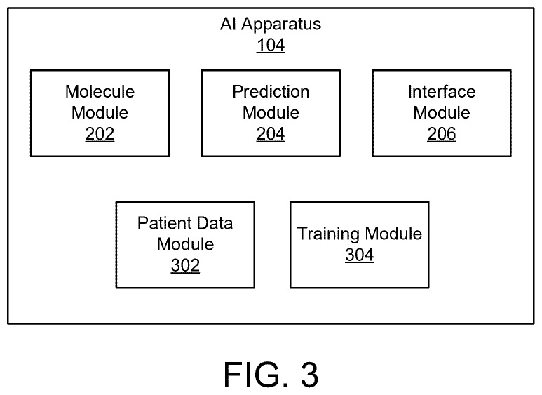

FIG. 3 illustrates one embodiment of an apparatus for AI-based side effect prediction. In one embodiment, the apparatus includes an instance of an AI apparatus 104. The AI apparatus 104, in one embodiment, includes a molecule module 202, a prediction module 204, and an interface module 206, as described above. In one embodiment, the AI apparatus 104 includes a patient data module 302 and a training module 304, which are described below.

The patient data module 302, in one embodiment, is configured to receive electronic health information, e.g., EHR data, for one or more patients and provide the electronic health information to a machine learning model. The prediction module 204 may use the electronic health information, in addition to the molecular structure information, to predict the one or more potential side effects of a drug.

In one embodiment, the patient data module 302 generates one or more personalized drug recommendations for the one or more patients. The recommendations or suggestions may include a recommendation regarding whether the patient should take the drug at all, a recommended dosage for the patient, and/or the like. One recommendation that the patient data module 302 may determine is whether the patient is a viable candidate for a drug test or study, e.g., whether the patient is likely to have a negative or positive or no reaction to the drug.

In one embodiment, the patient data module 302 and/or the prediction module 204 creates a joint embedding comprising the electronic health information for a patient and the molecular structure information to be analyzed by a machine learning model or multiple, different machine learning models. In one embodiment, the patient data module 302 and/or the prediction module 204 creates the joint embedding by concatenating the electronic health information for a patient with the molecular structure information.

To provide patient-specific recommendations, the patient data module 302 provides an architecture that extends the preliminary models with patient-specific embeddings extracted from EHR. By concatenating patient embeddings with molecular embeddings to construct a joint embedding, the patient data module 302 achieves a design that can learn to predict patient-specific side effects from EHR side effect annotations.

In one embodiment, in an effort to preserve the privacy of the patient and increase the security of the system, the patient data module 302 does not persistently store the electronic health information for the patient. Instead, the patient data module 302 may cache or store the electronic health information in volatile memory, may delete the electronic health information after it is analyzed or used, and/or the like. In one embodiment, the patient data module 302 may preprocess the electronic health information to remove personally identifiable information (PII) and/or other sensitive information that is not necessary for predicting whether the patient will suffer a side effect of a drug.

In one embodiment, the training module 304 is configured to train machine learning models to process molecular structure information and/or patient electronic health information. In one embodiment, the training module 304 trains machine learning models that map molecular structures and/or patient health information to predicted side effects.

In particular, in one embodiment, the training module 304 trains a random forest (RF) model and a graph-convolutional (GCN) model for each of the 27 SIDER side effects. In one embodiment, the random forests have 100 trees, and the graph convolutions have two convolution layers and a 128 dimensional output for each molecule. In one embodiment, the training module 304 trains GCNs for 75 epochs with a learning rate of 0.001.

In one embodiment, the training module 304 performs minimal hyperparameter tuning to avoid overfitting. In one embodiment, the training module 304 evaluates proof-of-concept models using the area under the curve of the receiver operator characteristic (ROC-AUC) and the area under the curve of the precision-recall curve (AUPRC). Building upon this, the training module 304 devises a design for an extended architecture combining patient information with molecular structure to yield personalized recommendations.

Table II shows one example of results for RF models. Table III shows one embodiment of results for GCNs. In such embodiments, the random baseline is the null model. Both models offer some predictive power, with GCNs slightly underperforming RFs.

TABLE II
Random Forest Metrics with Random Baselines.

| Side Effect | ROC-AUC | Random | AUPRC | Random |
|---|---|---|---|---|
| Hepatobiliary | 0.739 | 0.5 | 0.785 | 0.514 |
| Metabolism | 0.603 | 0.5 | 0.849 | 0.689 |
| Musculoskeletal | 0.576 | 0.5 | 0.785 | 0.685 |
| Gastrointestinal | 0.606 | 0.5 | 0.968 | 0.904 |
| Immune system | 0.603 | 0.5 | 0.874 | 0.707 |

TABLE III
Graph Convolution Metrics with Random Baselines.

| Side Effect | ROC-AUC | Random | AUPRC | Random |
|---|---|---|---|---|
| Hepatobiliary | 0.700 | 0.5 | 0.738 | 0.514 |
| Metabolism | 0.586 | 0.5 | 0.818 | 0.689 |
| Musculoskeletal | 0.565 | 0.5 | 0.740 | 0.685 |
| Gastrointestinal | 0.688 | 0.5 | 0.969 | 0.904 |
| Immune system | 0.591 | 0.5 | 0.807 | 0.707 |

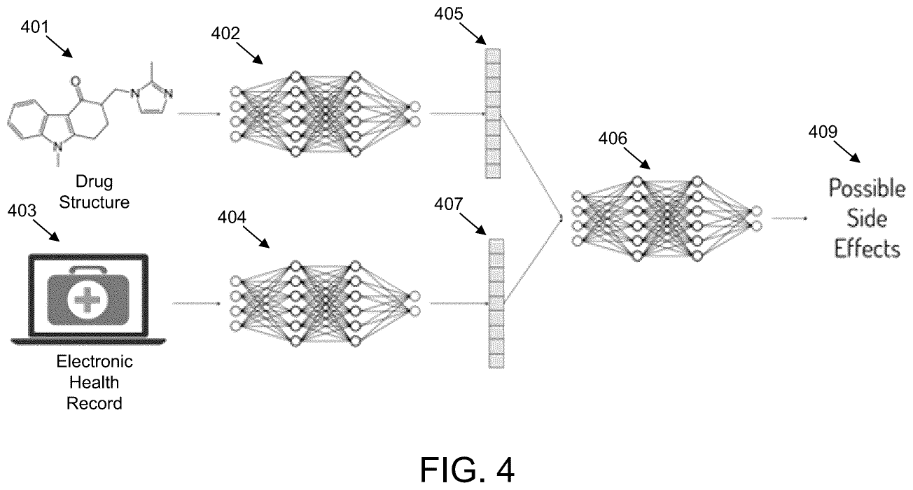

FIG. 4 illustrates one embodiment of a system architecture for the subject matter described herein. In particular, FIG. 4 shows the AI architecture for personalized side effect prediction. In one embodiment, a first machine learning model 402 analyzes or processes molecular structure information 401 for molecules associated with a drug to create a molecular embedding vector 405. In one embodiment, a second machine learning model 404 analyzes or processes electronic patient health information 403 to create a patient embedding vector 407. In one embodiment, a third machine learning model 406 analyzes or processes a combination of the molecular structure information 401 in the molecular embedding vector 405 and the electronic health information 403 in the patient embedding vector 407 to determine a personalized prediction and/or recommendation associated with potential side effects 409 of a drug for the user.

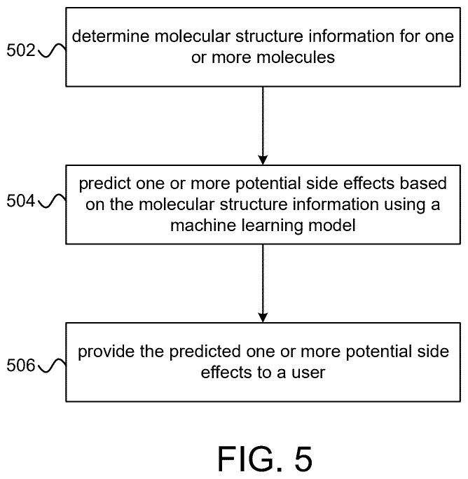

FIG. 5 illustrates one embodiment of a method for AI-based drug side effect prediction. The method may be performed by an information handling device 102, a server 108, an AI apparatus 104, a molecule module 202, a prediction module 204, and/or an interface module 206.

In one embodiment, the method determines 502 molecular structure information for one or more molecules, predicts 504 one or more potential side effects based on the molecular structure information using a machine learning model, and provides 506 the predicted one or more potential side effects to a user.

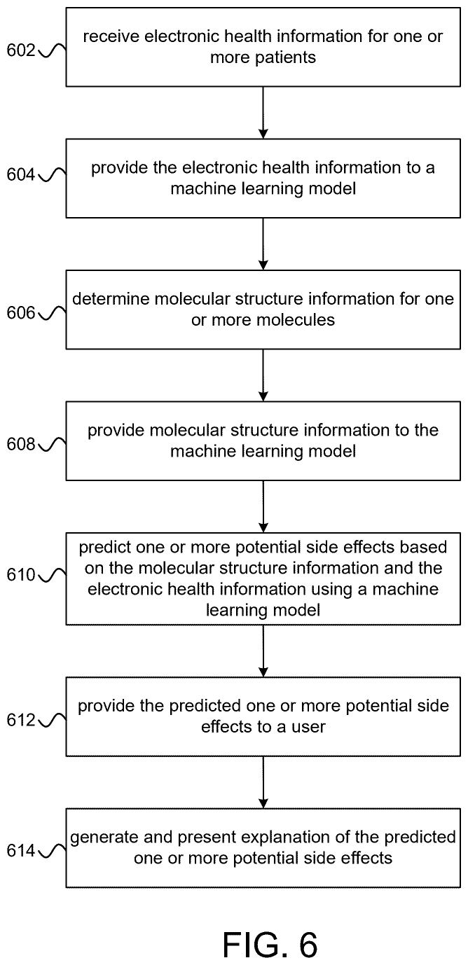

FIG. 6 illustrates one embodiment of a method for AI-based drug side effect prediction. The method may be performed by an information handling device 102, a server 108, an AI apparatus 104, a molecule module 202, a prediction module 204, an interface module 206, and/or a patient data module 302.

In one embodiment, the method receives 602 electronic health information for one or more patients and provides 604 the electronic health information to a machine learning model. In one embodiment, the method determines 606 molecular structure information for one or more molecules and provides 608 the molecular structure information to the machine learning model. In one embodiment, the method processes 610 the electronic health information and the molecular structure information to predict one or more potential side effects for the one or more patients and provides 612 the predicted one or more potential side effects to a user. In one embodiment, the method generates 614 and presents an explanation of the predicted one or more potential side effects.

Apparatuses, methods, program products, and systems are disclosed for AI-based drug side effect prediction. In one embodiment, an apparatus is configured to determine molecular structure information for one or more molecules, predict one or more potential side effects based on the molecular structure information using a machine learning model, and provide the predicted one or more potential side effects to a user.

In one embodiment, the apparatus is configured to receive electronic health information for one or more patients, provide the electronic health information to the machine learning model, and predict the one or more potential side effects based at least in part on the electronic health information for the one or more patients.

In one embodiment, the apparatus is configured to generate one or more personalized drug recommendations for the one or more patients. In one embodiment, the apparatus is configured to create a joint embedding comprising the electronic health information for a patient and the molecular structure information. In one embodiment, the apparatus is configured to create the joint embedding by concatenating the electronic health information for a patient with the molecular structure information.

In one embodiment, the apparatus is configured to receive the electronic health information for the one or more patients without persistently storing the electronic health information. In one embodiment, the machine learning model comprises a random forest model. In one embodiment, the random forest model comprises at least 100 trees. In one embodiment, the machine learning model comprises a graph-convolution model. In one embodiment, the graph-convolution model comprises at least two convolution layers and at least a 128 dimension output for each of the one or more molecules.

In one embodiment, the apparatus is configured to train the machine learning model using information describing molecules and one or more side effects associated with the molecules. In one embodiment, the apparatus is configured to group the information by molecules that have a same core structure.

In one embodiment, the apparatus is configured to train the machine learning model using side effect information for one or more drugs. In one embodiment, the one or more drugs comprise drugs that are approved by a regulatory organization. In one embodiment, the molecular structure information comprises strings of formatted textual representations of the one or more molecules.

In one embodiment, the apparatus is configured to convert the strings into vectorial representations of the one or more molecules using circular fingerprints. In one embodiment, each of the one or more molecules is represented as a sparse bit vector or a molecular graph. In one embodiment, the apparatus is configured to generate an explanation of the one or more potential side effects using a large language model.

In one embodiment, a system includes a first machine learning model configured to analyze molecular structure information for one or more molecules to create a molecular embedding vector, a second machine learning model configured to analyze electronic health information for one or more patients to create a patient embedding vector, and a third machine learning model configured to analyze a combination of the molecular embedding vector and the patient embedding vector to predict one or more potential side effects.

A method, in one embodiment, includes determining molecular structure information for one or more molecules, predicting one or more potential side effects based on the molecular structure information using a machine learning model, and providing the predicted one or more potential side effects to a user.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods, and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.” As used herein, “a member selected from the group consisting of A, B, and C and combinations thereof” includes only A, only B, only C, a combination of A and B, a combination of B and C, a combination of A and C or a combination of A, B and C.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
at least one memory; and
at least one processor coupled with the at least one memory and configured to cause the apparatus to:
determine molecular structure information for one or more molecules;
predict one or more potential side effects based on the molecular structure information using a machine learning model; and
provide the predicted one or more potential side effects to a user.

2. The apparatus of claim 1, wherein the at least one processor is configured to cause the apparatus to:
receive electronic health information for one or more patients;
provide the electronic health information to the machine learning model; and
predict the one or more potential side effects based at least in part on the electronic health information for the one or more patients.

3. The apparatus of claim 2, wherein the at least one processor is configured to cause the apparatus to generate one or more personalized drug recommendations for the one or more patients.

4. The apparatus of claim 2, wherein the at least one processor is configured to cause the apparatus to create a joint embedding comprising the electronic health information for a patient and the molecular structure information.

5. The apparatus of claim 4, wherein the at least one processor is configured to cause the apparatus to create the joint embedding by concatenating the electronic health information for a patient with the molecular structure information.

6. The apparatus of claim 2, wherein the at least one processor is configured to cause the apparatus to receive the electronic health information for the one or more patients without persistently storing the electronic health information.

7. The apparatus of claim 1, wherein the machine learning model comprises a random forest model.

8. The apparatus of claim 7, wherein the random forest model comprises at least 100 trees.

9. The apparatus of claim 1, wherein the machine learning model comprises a graph-convolution model.

10. The apparatus of claim 9, wherein the graph-convolution model comprises at least two convolution layers and at least a 128 dimension output for each of the one or more molecules.

11. The apparatus of claim 1, wherein the at least one processor is configured to cause the apparatus to train the machine learning model using information describing molecules and one or more side effects associated with the molecules.

12. The apparatus of claim 11, wherein the at least one processor is configured to cause the apparatus to group the information by molecules that have a same core structure.

13. The apparatus of claim 1, wherein the at least one processor is configured to cause the apparatus to train the machine learning model using side effect information for one or more drugs.

14. The apparatus of claim 13, wherein the one or more drugs comprise drugs that are approved by a regulatory organization.

15. The apparatus of claim 1, wherein the molecular structure information comprises strings of formatted textual representations of the one or more molecules.

16. The apparatus of claim 15, wherein the at least one processor is configured to cause the apparatus to convert the strings into vectorial representations of the one or more molecules using circular fingerprints.

17. The apparatus of claim 16, wherein each of the one or more molecules is represented as a sparse bit vector or a molecular graph.

18. The apparatus of claim 1, wherein the at least one processor is configured to cause the apparatus to generate an explanation of the one or more potential side effects using a large language model.

19. A system, comprising:
a first machine learning model configured to analyze molecular structure information for one or more molecules to create a molecular embedding vector;
a second machine learning model configured to analyze electronic health information for one or more patients to create a patient embedding vector; and
a third machine learning model configured to analyze a combination of the molecular embedding vector and the patient embedding vector to predict one or more potential side effects.

20. A method, comprising:
determining molecular structure information for one or more molecules;
predicting one or more potential side effects based on the molecular structure information using a machine learning model; and
providing the predicted one or more potential side effects to a user.
